(19) United States
(12) Patent Application Publication
(10) Pub. No.: US 2018/0344671 A1
(43) Pub. Date: Dec. 6, 2018

(54) Cysteamine as Neutrophil Elastase Inhibitor

(71) Applicant: Novabiotics Limited, Aberdeen (GB)

(72) Inventors: Deborah O'Neil, Aberdeen (GB); Douglas Fraser-Pitt, Aberdeen (GB)

(21) Appl. No.: 15/997,000

(22) Filed: Jun. 4, 2018

Related U.S. Application Data
(60) Provisional application No. 62/515,922, filed on Jun. 6, 2017.

(51) Int. Cl. A61K 31/145; A61K 31/194

(52) U.S. Cl. CPC A61K 31/145; A61K 31/194

(57) ABSTRACT

The present invention relates to compositions of cysteamine or a derivative thereof for use in the treatment or prophylaxis or a disease or condition associated with neutrophil elastase activity. The compositions can be administered, e.g., by inhalation, intravenously, or topically, as appropriate.

### FIELD OF THE INVENTION

The present invention relates to a composition comprising cysteamine or a derivative thereof for use in the treatment or prophylaxis of a disease or condition associated with neutrophil elastase activity. It also relates to methods of treatment or preventing a disease or condition associated with neutrophil elastase activity with the composition. In an aspect, cysteamine or a derivative thereof is used as a neutrophil elastase inhibitor and to a neutrophil elastase inhibitor comprising cysteamine or a derivative thereof.

### BACKGROUND TO THE INVENTION

Neutrophil elastase (NE; also known as leukocyte elastase, ELANE, ELA2, elastase 2, neutrophil elaszym, serine elastase, subtype human leukocyte elastase (HLE)) is a serine proteinase with broad substrate specificity. Secreted by neutrophils and macrophages during inflammation, it acts against bacteria as part of the innate host defense mechanism to kill/clear infectious agents. It is notable that NE activity is broad spectrum and non-specific, so host tissue is known to often be broken down as collateral damage.

Physiologically, NE is involved in the degradation of foreign materials ingested by neutrophils during phagocytosis. However, damage to connective tissue caused by leakage of enzymes is normally limited by proteinase inhibitors. It has been suggested that insufficient levels of these inhibitors leads to conditions such as pulmonary emphysema and elastase, in particular, has been implicated in abnormal lung connective tissue turnover.

The proteolytic activity of neutrophil elastase not only destroys pathogens but also degrades host matrix tissues by generating a localized protease/anti-protease imbalance. In humans, neutrophil elastase is well known to be involved in various acute and chronic inflammatory diseases, such as chronic obstructive pulmonary disease, emphysema, asthma, acute lung injury, acute respiratory distress syndrome, and cystic fibrosis. We have considered the regulation of production and activity of neutrophil elastase activity to represent a promising therapeutic approach we consider an important target for the development of novel selective inhibitors to treat these diseases.

### STATEMENTS OF THE INVENTION

According to a first aspect of the invention, there is provided a composition comprising cysteamine or a derivative thereof for use in the treatment or prophylaxis of a disease or condition associated with neutrophil elastase activity.

The present invention is predicated on the surprising finding that cysteamine, and/or derivatives thereof, have particular utility as inhibitors of neutrophil elastase activity.

In one embodiment the condition or disease is an inflammatory condition or disease, a genetic condition or disease, an autoimmune condition or disease or a cancer.

In one embodiment the condition or disease is a respiratory or pulmonary condition or disease.

In one embodiment the respiratory or pulmonary condition or disease is selected from the group consisting: cystic fibrosis, asthma, acute respiratory distress syndrome, chronic obstructive pulmonary disease, acute lung injury, emphysema, and lung cancer.

In one embodiment the autoimmune condition or disease is rheumatoid arthritis, IBD, or Crohns disease.

In one embodiment the genetic condition or disease is congenital neutropenia. The genetic condition or disease may be a cancer.

In one embodiment the cancer is lung cancer or colorectal cancer. The present invention is predicated on the association between neutrophil elastase activity and cancer proliferation.

In one embodiment the composition is a pharmaceutical composition further comprising a pharmaceutically acceptable carrier, excipient or diluent.

In one embodiment the composition is formulated for respiratory administration.

A further aspect the present invention provides a method of treating or preventing a condition or disease associated with neutrophil elastase activity comprising administering a pharmaceutically effective amount of a composition comprising cysteamine or a derivative thereof to a patient in need thereof.

Suitably, the condition or disease may be an inflammatory condition or disease, a genetic condition or disease, an autoimmune condition or disease or a cancer. Suitably, the condition or disease is a respiratory or pulmonary condition or disease. Suitably, the respiratory or pulmonary condition or disease is selected from the group consisting: cystic fibrosis, asthma, acute respiratory distress syndrome, chronic obstructive pulmonary disease, acute lung injury, emphysema, and lung cancer.

In another aspect, the present invention provides a method of preventing or treating any one or more of the group consisting of: cystic fibrosis, asthma, acute respiratory distress syndrome, chronic obstructive pulmonary disease, acute lung injury, emphysema and lung cancer in a patient comprising administering a composition comprising a pharmaceutically effective amount of cysteamine, or a derivative thereof to a patient.

In a further aspect there is provided use of cysteamine or a derivative as a neutrophil elastase inhibitor.

In yet another aspect there is provided a neutrophil elastase inhibitor comprising cysteamine or a derivative thereof.

### DETAILED DESCRIPTION

The present invention provides a composition comprising cysteamine or a derivative thereof for use in the treatment or prophylaxis of a disease or condition associated with neutrophil elastase activity.

The term “composition comprising cysteamine and/or a derivative thereof” encompasses the use of cysteamine and/or a derivative thereof optionally in combination with other undefined ingredients. The term “composition comprising” where used may alternately be substituted with “consisting essentially of” or “consisting of” in discussions herein.

In one embodiment cysteamine is cysteamine free base.

Cysteamine as employed herein refers to the molecule NH₂—CH₂—CH₂—SH.

Examples of cysteamine derivatives include: 2-methylthio ethylamine (cinnamate), 2-methyl thio ethylurea, N-(2-methylthio ethyl) p-acetamido benzamide, 2-aminoethanethiol, N-(2-methylthio ethyl)p-acetamido benzenesulfonamide, N-(2-propylthioethyl)-p-methoxy benzamide, N-(butylthio ethyl) nicotinamide, N-(2-dodecylthio ethyl) p-butoxybenzamide, N-(2-methylthio ethyl) p-toluenesulfonamide, N-(2-isopropylthio ethyl) propionamide, N-(2-octylthio ethyl) acetamide, N-(2-butylthio ethyl) methanesulfonamide, N-(2-isopentylthio ethyl)butane, bis 1,4-(2-acetamido ethylthio), 2,3-butanediol, 2-hexadecylthio ethylamine hydrochloride, 2-allylthio ethylamine malate, 9-octadecene 2-ylthio ethylamine hydrochloride, 2-dodecylthio ethylamine hydrochloride, 2-isopentylthio ethylamine mandelate, 2-octadecylthio ethylamine salicylate, 2-β-hydroxyethyl thio ethylurea, 2-β-hydroxyethylthio ethylamine hydrochloride, 2-(2,3-dihydroxy propylthio)ethylamine p-toluenesulfonate, 2-(2-hydroxy propylthio)ethylamine oxalate, N-(2-methylthio ethyl)phenylacetamide, 2-(2,2-dimethoxy ethylthio) ethylamine hydrochloride, 2-(2,2-dimethoxy ethylthio) ethylamine undecylenate, 2-(2,2-diethoxy ethylthio) ethylamine undecylenate, 2-(2,2-diethoxy ethylthio)ethylamine acetate, 2-undecenylthio ethylamine, 2-β-ureidoethylthio ethylamine hydrochloride, 2-β-acetamidoethylthio ethylamine tropate, 2,2′-thio diethylamine fumarate, 2,2′-thio diethylurea, 3-β-aminoethylthio propylamine hydrochloride, S-β-ureidoethyl thiocarbamate, 2-ethoxycarbonylthio ethylamine hydrochloride, 2-dimethylamino carbonylthio ethylamine sulfate, 2-butoxycarbonyl methylthio ethylurea, 2-ethyloxycarbonylmethylthio ethylamine hydrochloride, 6-β-aminoethylthio hexanoate of methyl hydrochloride, 5-β-aminoethylthio pentanoic acid, 2-phenylthio ethylamine dihydrogen phosphate, 2-p-t-butylphenylthio ethylamine trichloracetate, 2-p-methoxyphenylthio ethylamine ditartrate, 2-tolylthio ethylamine hydrobromide, 2-(1-biphenyl thio) ethylamine hydrochloride, 2-N-pentachlorophenylthio ethyl acetamide, 2-benzylthio ethylamine malate, 2-benzylthio ethylamine nicotinate, 2-benzylthio 2-methyl propylamine hydrochloride, 2-benzylthio propylamine lactate, N-(2-benzylthio ethyl)nicotinamide hydrochloride, N-(2-benzylthio ethyl) 10-undecene amide, N-(2-benzylthio ethyl) hexadecanamide, S-β-aminoethyl mercaptobutyric acid, N-(2-benzylthio ethyl)formamide, N-(2-benzylthio ethyl)phenylacetamide, N-(2-(2,6-dimethyl phenyl)ethyl] hexanamide, 2-o-aminophenylthio ethylamine succinate, N-(2-benzylthio ethyl) glutamine, S-β-aminoethyl mercapto acetic acid (3-S-β-aminoethyl) mercapto propionic acid, (3-S-.gamma-amino propyl) mercapto acetic acid, S(2-p-methoxybenzamido ethyl) mercapto 2-(2-naphtyl methylthio) ethylamine hydrochloride, 2-(2-naphtyl methylthio) ethylamine disuccinate, (2-thenyl) 2-thio ethylamine hydrobromide, 2-N-acetyl (2-thenylthio-ethylamine, 2-o-chlorobenzylthio ethylamine hydrochloride, 2-p-chlorobenzylthio ethylamine glycolate, 2-o-fluorobenzylthio ethylamine hydrochloride, 2-furfurylthio ethylamine hydrochloride, 2-tetrahydrofurfurylthio ethylamine p-amino-benzoate, 2-β-phenylethylthio ethylamine glutamate, 2-diphenylmethylthio ethylamine hydrochloride, 2-triphenyl methylthio ethylamine hydrochloride hemihydrate, 2-(2-pyridyl ethylthio)ethylamine hydrochloride, 2-(2-p-toluene sulfonamido ethylthio) pyridine N-oxide, 2-β-aminoethylthiomethyl pyridine N-oxide dihydrochloride, 2-β-aminoethylthio pyridine N-oxide hydrochloride, 2,4-dichloro 2-benzylthio ethylamine aspartate, N-[2-(3,4-dichloro benzylthio)ethyl] butyramide, N-[2-(2,6-dichloro benzylthio)ethyl] dodecanamide, N-[2-(3,5-dichloro benzylthio)ethyl] trifluoroacetamide hydrochloride, 2-p-ethoxybenzylthio ethylamine hydrochloride, N-[2-m-fluorobenzylthio ethyl] chloroacetamide, 2-p-bromobenzylthio ethylamine succinate, 2-(3,4-dimethoxy benzylthio)ethylamine malate, 2-(3,4-methylenedioxy benzylthio)ethylamine hydrochloride, 2-(2,4-dichloro cetylthio) ethylamine, 2 (3,4,5-trimethoxy benzylthio) ethylamine hydrocinnamate, 2-p-methoxy benzylthio ethylamine salicylate, 2-o-methylbenzylthio ethylamine phenyl-acetate, N-[2-p-dimethylaminobenzylthio ethyl] methane-sulfonamide, 2-p-phenoxybenzylthio ethylamine hydrochloride, 2-β-aminoethylthio pyridine hydrochloride, 2-benzylthio ethylamine citrate, N-[2-benzylthio ethyl] 2,4-dihydroxy 3,3-dimethyl butyramide, N-(2-benzylthio ethyl) 6,8-dihydroxy 7,7-dimethyl 5-oxo 4-aza octanamide, N-[2-(2-pyridyl thio)ethyl] propionamide, 2-(2-pyridyl methylthio)ethylamine dihydrochloride, 2-benzylthio ethylamine pantothenate, S-(β-acetamidoethyl)mercaptoacetate of beta.-morpholinoethyl, S-(β-henylacetamidoethyl) mercaptoacetate N′-methyl 2-piperazino ethyl, S-(β-ureidoethyl)mercaptoacetate of beta.-pyrrolidino-ethy, S-(β-trifluoroacetamidoethyl)-βmercapto-propionate of β-dimethylaminoethyl, 2-p-nitrobenzylthio ethylamine crotonate, 2-β-morpholinocarbonyl ethylthio ethylamine hydrochloride, N,N-di(hydroxyethyl)S-(β-benzamido-ethyl) mercapto-acetamido, N[2-N′-methyl piperazino carbonylthio ethyl] acetamide, 2-(1-naphthyl thio)ethylamine hydrochloride, N-(3-β-ureidoethylthio propyl) succinamic acid, 3-allylthio propylamine, 3-(2,2′-dimethoxy ethylthio)propylamine, 3-(2,2′-dimethoxy ethylthio) propylamine sulfate, S-β-aminoethylmercapto acetic acid, the hydrochloride of S-β-aminoethyl mercapto acetic acid, N-(2-benzylthioethyl)acetamide, N-(2-benzylthioethyl) propionamide, N-(2-benzylthioethyl)butyramide, N-(2-benzylthioethyl) methanesulfonamide, N-(2-benzylthioethyl) ethanesulfonamide, N-(2-benzylthioethyl-propanesulfonamide, N-(2-benzylthioethyl)butanesulfonamide, S-(2-p-acetamidobenzenesulfonamido ethyl) mercapto acetic acid, S-(2-p-acetamidobenzamido ethyl) mercapto acetic acid, N-(2-thenylthioethyl) acetamide, 2-benzylthio propylamine, 2-benzylthio 2-methyl propylamine, 2-(2-p-toluenesulfonamido ethylthio) pyridine N-oxide, S-(2-p-butoxybenzamidoethyl)mercapto acetic acid, 2-t-butylthio ethylamine hydrochloride, 2-methoxycarbonyl methylthio ethylamine hydrochloride, 2-ethoxycarbonylmethylthio ethylamine hydrochloride, 2-propoxycarbonylmethyl thio ethylamine hydrochloride, 2-butoxycarbonylmethylthio ethylamine hydrochloride, 2,2′-thio diethylamine dihydrochloride, 3-(2-aminoethylthio)alanine hydrochloride, 2-benzylthio ethylammonium diacid phosphate, 2-methylthio ethylamine, N-(methylthioethyl) p-acetamidobenzamide, N-(2-methylthioethyl)nicotinamide, N-(2-methylthioethyl)benzamide, N-(2-methylthioethyl) p-butoxybenzamide, N-(2-methylthioethyl) butyramide, N-(2-methylthioethyl) propionamide, N-(2-methylthioethyl) acetamide, N-(2-methylthioethyl) butanesulfonamide, N-(2-octylthioethyl) methanesulfonamide, 2-cetylthio ethylamine hydrochloride, 2-(2-hydroxyethylthio) ethylamine hydrochloride, 2-methylthio ethylamine phenylacetate, and 2-methylthio ethylamine undecylenate, cystamine.

Cysteamine or derivatives thereof can exist in different forms, such as free acids, free bases, esters and other prodrugs, salts, and tautomers. For example, the invention can include all variant forms of the compounds. In one embodiment the derivative is cystamine and the salt thereof is cystamine dihydrochloride. Thus, the invention encompasses the salt or pro-drug of cysteamine or a derivative thereof. In one embodiment cysteamine is cysteamine bitartrate. In one embodiment the cysteamine derivative is cystamine or a salt or pro-drug thereof.

As employed herein neutrophil elastase activity refers to the activity of neutrophil elastase, typically in inflammation (i.e, where this an infiltration of neutrophils to the affected tissue and activation of neutrophils at that site, e.g., therefore with consequent increase in neutrophil elastase activity, e.g., above any normal background in a non-inflamed state). Typically, a disease or condition associated with neutrophil elastase activity refers to the deviation from normal activity of neutrophil elastase, such as a condition with neutrophilic inflammation.

In one embodiment, the disease or condition associated with neutrophil elastase activity is a disease or condition caused by neutrophil elastase activity. For example, the over or under-expression of neutrophil elastase, typically the overexpression of neutrophil elastase, excess release of neutrophil elastase, the expression of mutated neutrophil elastase, or the occurrence of neutrophil elastase in an erroneous location. In some examples the disease or condition is enhanced due to the presence of too little neutrophil elastase inhibitor, for example, too little alpha-1 antitrypsin (A1AT).

In one embodiment the disease or condition associated with neutrophil elastase, activity is downstream of the over or under-expression of neutrophil elastase, the expression of mutated neutrophil elastase or the occurrence of neutrophil elastase in an erroneous location. In one embodiment according to the invention the condition or disease associated with neutrophil elastase activity is an inflammatory condition or disease, a genetic condition or disease, an autoimmune condition or disease or a cancer. Inflammatory condition or disease as employed herein refers to diseases or conditions in which the normal base line inflammation is altered.

“Genetic condition or disease” as employed herein refers to a disease or condition in which the DNA or genetic machinery is defective in somatic and/or germline cells.

“Autoimmune condition or disease” as employed herein refers to the system of immune responses of an organism against its own healthy cells and tissues, as is known in the art. Any disease or condition that results from such an aberrant immune response is an autoimmune disease or condition.

“Cancer” as employed herein refers to a group of diseases or conditions involving abnormal cell growth with the potential to invade or spread to other parts of the body, as is known in the art.

In one embodiment the condition or disease is a respiratory or pulmonary condition or disease. “Respiratory condition or disease” as employed herein refers to a disease or condition affecting the respiratory system. “Pulmonary condition or disease” refers to a condition or disease affecting the lungs. In one embodiment, the respiratory or pulmonary condition or disease is selected from the group consisting: cystic fibrosis, asthma, acute respiratory distress syndrome, chronic obstructive pulmonary disease, acute lung injury, emphysema, and lung cancer.

In one embodiment the condition or disease is cystic fibrosis or COPD. In one embodiment the condition or disease is idiopathic pulmonary fibrosis (IPF).

The compositions of the invention are, e.g., potent anti-inflammatories with particular benefit where there is a direct route of delivery (for example, where the condition or disease is in the lung and the composition can be delivered via inhaler, nebuliser or similar). However, the compositions of the invention may also be useful in the treatment of other conditions including, but not limited to, rheumatoid arthritis, congenital neutropenia or cancer. In one embodiment, the compositions of the invention are useful in treating the symptoms of respiratory/lung disease, particularly in cystic fibrosis or COPD.

The term “treatment” relates to the effects of cysteamine or derivative thereof imparting a benefit to a patient afflicted with a condition or disease, including an improvement in the condition of the patient or delay in disease progression.

The term “prophylaxis” as employed herein refers to the prevention of a condition or disease aimed at stopping the condition or disease developing or progressing, such as a condition or disease associated with neutrophil elastase.

In one embodiment, there is provided a method of treating or preventing a condition or disease associated with neutrophil elastase activity in a subject comprising administering to said subject a therapeutically effective amount of a composition according to the invention.

By an “effective” amount or “therapeutically effective amount” is meant an amount of one or more active substances which, within the scope of sound medical judgment, is sufficient to provide a desired effect without excessive toxicity, irritation, allergic response, or other problem or complication, commensurate with a reasonable benefit/risk ratio.

In one embodiment, there is provided use of cysteamine or a derivative thereof as a neutrophil elastase inhibitor.

In one embodiment, the use is in vivo or ex vivo.

In one embodiment, there is provided a neutrophil elastase inhibitor comprising cysteamine or a derivative thereof.

The composition of the invention may be administered in the form of a pharmaceutically acceptable salt. The pharmaceutically acceptable salts of the present invention can be synthesized from the parent compound which contains a basic or acidic moiety by conventional chemical methods. Generally, such salts can be prepared by reacting the free acid or base forms of cysteamine or derivative thereof with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the composition of the invention wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, bitartrate, butyrate, citrate, camphorate, camphorsulfonate, cyclopentanepropionate, digluconate, dihydrochloride, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides, and/or others.

The invention includes prodrugs for the active pharmaceutical species of cysteamine or a cysteamine derivative, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

A further aspect of the invention provides a pharmaceutical composition comprising a pharmaceutically effective amount of cysteamine and/or a derivative thereof.

The composition may also include a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

In the method of the invention the composition is preferably administered via inhalation.

Mammals, birds and other animals may be treated by the compositions or methods described herein. Such mammals and birds include humans, dogs, cats and livestock, such as horses, cattle, sheep, goats, chickens, turkeys, and the like.

To achieve the desired effect(s), the composition, a variant thereof or a combination thereof, may be administered as single or divided dosages, for example, of at least about 0.001 mg/kg to about 500 to 750 mg/kg, of at least about 0.001 mg/kg to about 300 to 500 mg/kg, at least about 0.1 mg/kg to about 100 to 300 mg/kg or at least about 1 mg/kg to about 50 to 100 mg/kg of body weight or at least about 1 mg/kg to about 20 mg/kg of body weight, although other dosages may provide net beneficial results. The amount administered will vary depending on various factors including, but not limited to, the cysteamine or cysteamine derivative chosen, the disease, the weight, the physical condition, the health, the age of the mammal, whether prevention or treatment is to be achieved.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the invention may be essentially continuous over a pre-selected period of time or may be in a series of spaced doses. Both local and systemic administration is contemplated.

To prepare one example of the composition, cysteamine and/or a derivative thereof is synthesized or otherwise obtained, purified as necessary or desired, and then lyophilized and stabilized. The composition can then be adjusted to the appropriate concentration and optionally combined with other agents.

Thus, one or more suitable unit dosage forms comprising the therapeutic composition of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. Cysteamine of a derivative thereof may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well-known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

The active agents of the present invention may be administered by any suitable route known to those skilled in the art, preferably in the form of a pharmaceutical composition adapted to such a route, and in a dose effective for the treatment intended. The active compounds and composition may, for example, be administered parenterally, orally, intranasal, intrabronchial, enterally, transdermally, sublingually, rectally, vaginally, ocularly, or topically. Both local and systemic administration is contemplated.

When the therapeutic compositions of the invention are prepared for inhalation, they are generally combined with a pharmaceutically acceptable carrier, diluent or excipient to form a pharmaceutical formulation, or unit dosage form. For inhalation, cysteamine or cysteamine derivative may be present as a powder, a granular formation, a solution, a suspension, an emulsion or in a natural or synthetic polymer for inhalation. For oral administration, the active ingredients may also be presented as a bolus, electuary or paste. Orally administered therapeutic compositions of the invention can also be formulated for sustained release, e.g., cysteamine can be coated, micro-encapsulated, or otherwise placed within a sustained delivery device. The total active ingredients in such formulations comprise from 0.1% to 99.9% by weight of the formulation. For oral administration, the pharmaceutical composition may be in the form of; for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose; mannitol, corn starch or potato starch; with binders such as crystalline cellulose, cellulose derivatives, acacia, corn starch or gelatins; with disintegrators such as corn starch, potato starch, or sodium carboxymethylcellulose; and with lubricants such as talc or magnesium stearate. The active ingredient may also be administered by injection as a composition wherein, for example, saline, dextrose or water may be used as a suitable carrier.

The products of the invention may also find application as/in an oral formulation wherein the product is formulated in a carrier, for example selected from films, tapes, gels, microspheres, lozenges, chewing gum, dentrifices, and mouthwash.

The products of the invention are preferably administered to the respiratory tract. Thus, the present invention also provides aerosol pharmaceutical formulations comprising a product of the invention. Also provided is a nebuliser or inhaler containing a product of the invention.

Pharmaceutical formulations containing the therapeutic composition of the invention can be prepared by procedures known in the art using well-known and readily available ingredients. For example, formulated with common excipients, diluents, or carriers, and formed into tablets, capsules, solutions, suspensions, powders, aerosols, and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatine, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones, montmorillonites, and the like.

For example, tablets or caplets containing the composition of the invention can include buffering agents such as calcium carbonate, magnesium oxide, and magnesium carbonate. Suitable buffering agents may also include acetic acid in a salt, citric acid in a salt, boric acid in a salt, and phosphoric acid in a salt. Caplets and tablets can also include inactive ingredients such as cellulose, pregelatinized starch, silicon dioxide, hydroxyl propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatine capsules containing cysteamine or derivative thereof can contain inactive ingredients such as gelatine, microcrystalline cellulose, sodium lauryl sulphate, starch, talc, and titanium dioxide, and the like, as well as liquid vehicles such as polyethylene glycols (PEGs), and vegetable oil. Moreover, enteric-coated caplets or tablets containing cysteamine or cysteamine derivative are designed to resist disintegration in the stomach and dissolve in the more neutral to alkaline environment of the duodenum.

The therapeutic composition of the invention can also be formulated as elixirs or solutions for convenient oral administration or as solutions appropriate for parenteral administration, for instance by intramuscular, subcutaneous, intraperitoneal or intravenous routes. The pharmaceutical formulations of the invention can also take the form of an aqueous or anhydrous solution or dispersion, or alternatively the form of an emulsion or suspension or salve.

For the purposes of parenteral administration (“parenteral” as used herein, refers to modes of administration which include intravenous, intramuscular, enteral, intraperitoneal, intrasternal, subcutaneous and intraarticular injection and infusion of which intravenous (including continuous intravenous administration) is most preferred) solutions in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Alternatively, the products of the invention can be administered in the form of a suppository or pessary, or they may be applied topically in the form of a gel, hydrogel, lotion, solution, cream, ointment or powder. The products of the invention may be dermally or transdermally administered, for example, by use of a skin patch, depot or subcutaneous injection. They may also be administered by pulmonary or rectal routes.

Thus, the therapeutic compositions may be formulated for parenteral administration (e.g. by injection, for example, bolus injection or continuous infusion) and may be presented in unit dose form in ampules, pre-filled syringes, small volume infusion containers or in multi-dose containers. The active ingredients may form suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution for constitution with a suitable vehicle, e.g. sterile, pyrogen-free water before use.

These formulations can contain pharmaceutically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils, and polysiloxanes.

Solvents or diluents comprising cysteamine or cysteamine derivative may include acid solutions, dimethylsulphone, N-(2-mercaptopropionyl) glycine, 2-n-nonyl-1,3-dioxolane and ethyl alcohol. Preferably the solvent/diluent is an acidic solvent, for example, acetic acid, citric acid, boric acid, lactic acid, propionic acid, phosphoric acid, benzoic acid, butyric acid, malic acid, malonic acid, oxalic acid, succinic acid, or tartaric acid.

Additionally, the compositions may be formulated as sustained release dosage forms and the like. The formulations can be so constituted that they release cysteamine or a derivative thereof, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices, and the like.

For topical administration, the active agents may be formulated as is known in the art for direct application to a target area. Forms chiefly conditioned for topical application take the form, for example, of creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, cysteamine or a derivative thereof, can be delivered via patches or bandages for dermal administration. Alternatively, the composition can be formulated to be part of an adhesive polymer, such as polyacrylate or acrylate/vinyl acetate copolymer. For long-term applications, it might be desirable to use microporous and/or breathable backing laminates, so hydration or maceration of the skin can be minimized. The backing layer can be any appropriate thickness that will provide the desired protective and support functions. A suitable thickness will generally be from about 10 microns to about 200 microns.

Pharmaceutical formulations for topical administration may comprise, for example, a physiologically acceptable buffered saline solution containing between about 0.001 mg/ml and about 100 mg/ml, for example between 0.1 mg/ml and 10 mg/ml, of cysteamine or cysteamine derivative.

Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or coloring agents. Cysteamine or cysteamine derivative can also be delivered via iontophoresis, e.g., as disclosed in U.S. Pat. Nos. 4,140,122; 4,383,529; or 4,051,842. The percentage by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with cysteamine or derivative thereof in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

Cysteamine or cysteamine derivative may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerine or sucrose and acacia; and mouthwashes comprising the composition of the present invention in a suitable liquid carrier. Alternatively, the active ingredients may be formulated as a film strip or buccal tablet, which may or may not be dissolvable.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The products of the invention can also be administered intranasally or by inhalation and are conveniently delivered in the form of a dry powder inhaler or an aerosol spray presentation from a pressurized container, pump, spray, atomizer, nebulizer, with or without the use of a suitable propellant.

The compositions of the invention can also be administered to the respiratory tract. For administration by inhalation or insufflation, the composition may take the form of a dry powder, for example, a powder mix of the therapeutic agent and a suitable powder base such as lactose or starch. Cysteamine or a derivative thereof can also be administered in an aqueous solution when administered in an aerosol or inhaled form. Thus, other aerosol pharmaceutical formulations may comprise, for example, a physiologically acceptable buffered saline solution containing between about 0.001 mg/ml and about 100 mg/ml for example between 0.1 and 100 mg/ml, such as 0.5-50 mg/ml, 0.5-20 mg/ml, 0.5-10 mg/ml, 0.5-5 mg/ml or 1-5 mg/ml cysteamine or cysteamine derivative specific for the indication or disease to be treated.

The above mentioned active agents may be administered as free or fixed combinations. Free combinations may be provided as combination packages containing all the active agents in free combinations. Fixed combinations are often tablets or capsules. The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

Throughout the description and claims of this specification, the words “comprise” and “contain” and variations of the words, for example “comprising” and “comprises”, means “including but not limited to”, and is not intended to (and does not) exclude other moieties, additives, components, integers, or steps.

Throughout the description and claims of this specification, the singular encompasses the plural, unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE FIGURES

FIG. 1: Cysteamine can form mixed disulphides with susceptible cysteines. A process known as cysteaminylation. This mixed disulphide is analogous to a lysine side chain, and this reaction can change the overall properties of a protein and is a principal behind the therapeutic activity of cysteamine in the metabolic condition, cystinosis.

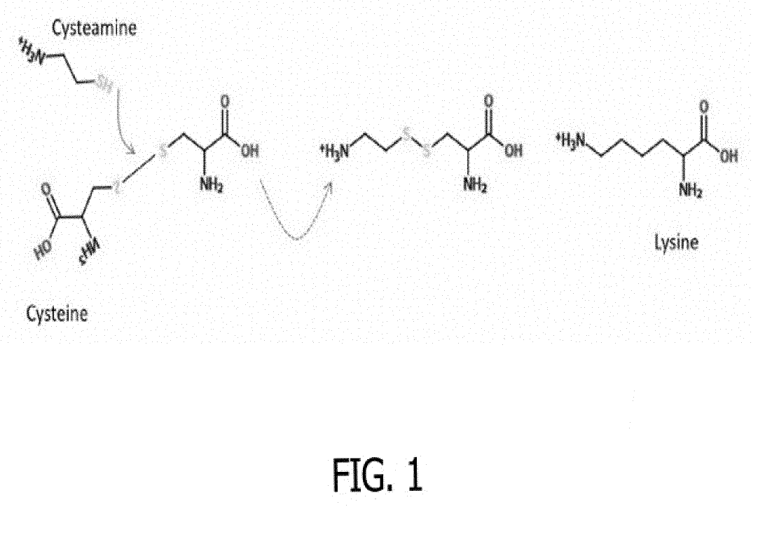

FIG. 2: Both cysteamine free base and cysteamine bitartrate effected human neutrophil elastase activity as measured fluorometrically using the neutrophil elastase inhibitor screening kit (Sigma). Cystamine dihydrochloride had minimal effect (data not shown).

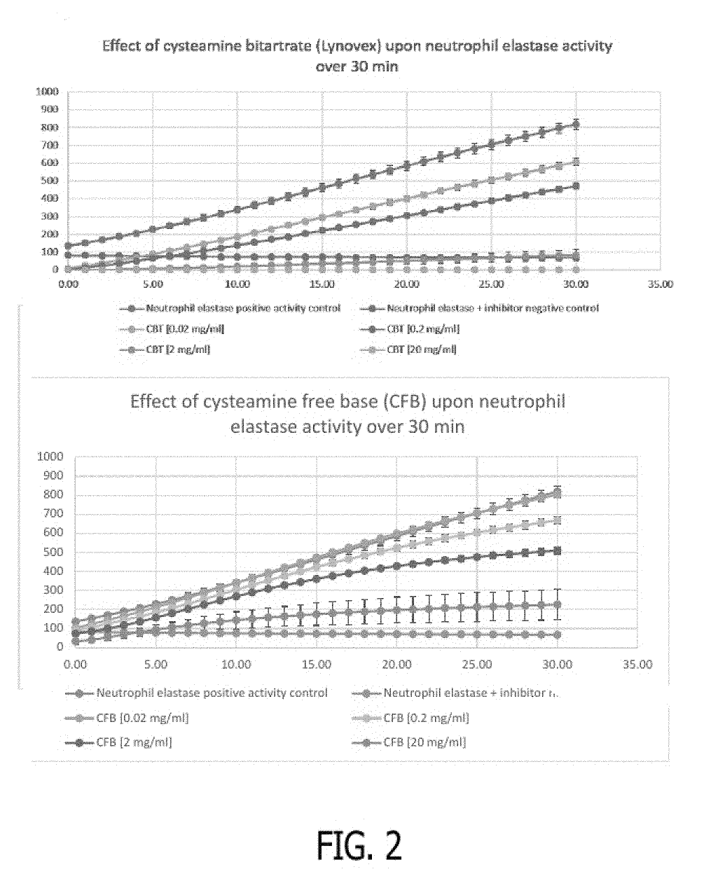

FIG. 3: The banding pattern and relative staining intensity of recombinant human neutrophil elastase is altered by the addition of cysteamine bitartrate and cysteamine free base. Cystamine dihydrochloride has less of an effect. This 8-16% gradient Tri-Glycine SDS gel is loaded with a molecular weight marker and: 1. Recombinant human neutrophil elastase [2.5 μg total]; 2. Recombinant human neutrophil elastase [2.5 μg total] and cysteamine bitartrate [2.5 μg total]; 3. Recombinant human neutrophil elastase [2.5 μg total] and cysteamine bitartrate [5 μg total]; 4. Recombinant human neutrophil elastase [2.5 μg total] and cysteamine free base [2.5 μg total]; 5. Recombinant human neutrophil elastase [2.5 μg total] and cysteamine free base [5 μg total]; 6. Recombinant human neutrophil elastase [2.5 μg total] and cystamine dihydrochloride [2.5 μg total]; 7. Recombinant human neutrophil elastase [2.5 μg total] and cystamine dihydrochloride [5 μg total].

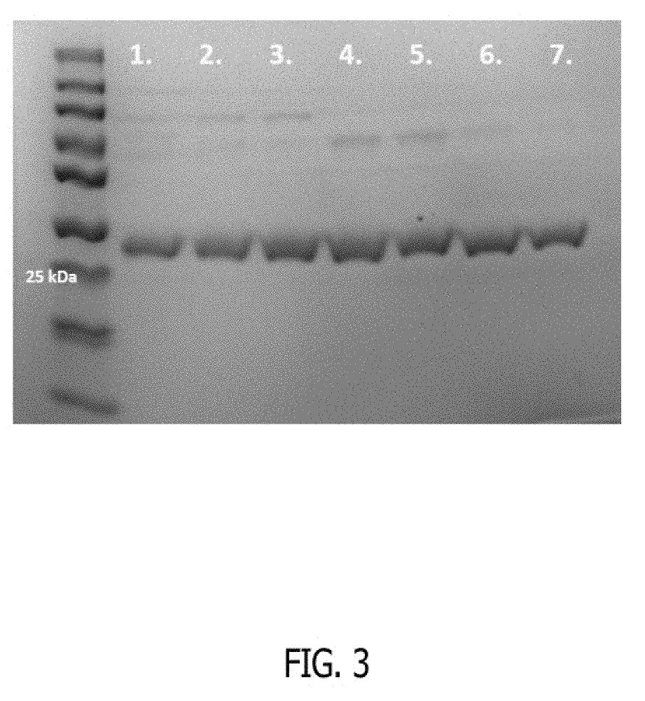

### EXAMPLE 1

Methods.

Neutrophil Elastase Inhibitor Screening Kit is available from Sigma-Aldrich, catalogue number MAK213. This kit measures neutrophil elastase activity by detecting the hydrolysis and release of a fluorescent product. It allows for the screening of unknown substances for inhibitory activity and compares with a positive inhibitor control.

Cysteamine free base, cysteamine bitartrate, and cystamine dihydrochloride were prepared in the assay buffer provided at four times the final testing concentration and combined with enzyme and substrate as appropriate and as defined in the full protocol. Samples were prepared in 96 well microtiter plates with black well walls. Background fluorescence can be determined by omitting the neutrophil elastase from a sample blank control.

Fluorescence was measured at 400ex/505em nm on a Biotek Synergy plate reader over 30 min at an incubation temperature of 37° C.

Relative inhibition is determined by the following equations:

Slope=(Fluorescence 2−Fluorescence 1)/(Time 2−Time 1)=delta fluorescence/minute

% Relative Inhibition=((SlopeEC−SlopeSM)/SlopeEC))×100

Where: SlopeSM=the slope of the sample inhibitor (cysteamine and derivatives) & SlopeEC=the slope of the Enzyme control.

SDS PAGE.

Recombinant proteins were treated with or without cysteamine and its derivatives at the concentrations stated in the figures below for 1 h at 37° C. prior to addition of 2× sample buffer and electrophoretic separation on pre-cast 8-16% tris-glycine SDS polyacrylamide gradient gel (Life Technologies) under a constant voltage of 120V in tris-glycine running buffer until the dye front reached the end of the gel. Samples were not heated prior to loading to minimize denaturation of disulphide bonds on the denaturing gel. Precision Plus (Bio-Rad) prestained protein standards were used alongside samples. After electrophoresis gels were carefully removed from the plastic cassettes and washed in sterile distilled water to remove SDS 3× for 5 min. Protein bands were visualized using coomassie-based Simply Blue™ Safe Stain (Life Technologies) staining for 1 h at room temperature. The gel was washed overnight in sterile distilled water, and for a further hour in fresh water prior to photographing.

TABLE 1
Cysteamine bitartrate inhibits neutrophil elastase activity

| Cysteamine bitartrate | Relative inhibition |
|---|---|
| concentration [μg/ml] | of NE activity (%) |
| 0 | 0 |
| 20 | 12.526 |
| 200 | 30.689 |
| 2000 | 86.639 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A composition comprising cysteamine or a derivative thereof for use in the treatment or prophylaxis of a disease or condition associated with neutrophil elastase activity.

2. The composition according to claim 1 wherein the disease or condition is selected from the group consisting: inflammatory conditions or diseases including COPD and cystic fibrosis; genetic conditions or diseases including congenital neutropenia; autoimmune conditions including rheumatoid arthritis; and cancers including lung cancer and colorectal cancer.

3. The composition according to claim 1 wherein the condition or disease is a respiratory condition or disease.

4. The composition according to claim 3 wherein the respiratory condition or disease is selected from the group consisting: cystic fibrosis, asthma, acute respiratory distress syndrome, chronic obstructive pulmonary disease, acute lung injury, emphysema and lung cancer.

5. The composition according to claim 1 formulated for respiratory administration.

6. The composition according to claim 1, wherein said composition is a pharmaceutical composition comprising a pharmaceutically acceptable carrier, excipient or diluent.

7. A method of treating or preventing a disease or condition associated with neutrophil elastase activity comprising administering a pharmaceutically effective amount of a composition comprising cysteamine or a derivative thereof to a patient in need thereof.

8. Use of cysteamine or a derivative thereof as a neutrophil elastase inhibitor.

9. (canceled)
